(19) United States
(12) Patent Application Publication
(10) Pub. No.: US 2018/0360521 A1
(43) Pub. Date: Dec. 20, 2018

(54) ELECTROSURGICAL SYSTEM AND ELECTROSURGICAL INSTRUMENT FOR USE IN A SYSTEM

(71) Applicant: OLYMPUS WINTER & IBE GMBH, Hamburg (DE)

(72) Inventors: Carlos GROTH, Berlin (DE); Christopher SPRENGER, Berlin (DE)

(73) Assignee: OLYMPUS WINTER & IBE GMBH, Hamburg (DE)

(21) Appl. No.: 15/780,911

(22) PCT Filed: Dec. 1, 2016
(86) PCT No.: PCT/EP2016/079453

§ 371 Date: Jun. 1, 2018

(30) Foreign Application Priority Data
Dec. 1, 2015 (DE) 10 2015 223 955.1

(51) Int. Cl. A61B 18/12; A61B 17/32; A61B 18/18

(52) U.S. Cl. CPC A61B 18/1206; A61B 17/320068; A61B 2018/00994; A61B 2018/126; A61B 18/1815

(57) ABSTRACT

An electrosurgical system and a method for operating an electrosurgical instrument comprises an electrosurgical instrument, a generator which is designed to operate the electrosurgical instrument, a memory element on which is stored at least one piece of operation-relevant information representing at least one operating instruction for the electrosurgical instrument, and a display which is designed to display operation relevant information, wherein the system is designed to transmit the operation relevant information to the display.

The present invention relates to an electrosurgical system, a method for operating an electrosurgical instrument, an electrosurgical instrument for use in an electrosurgical system, and a generator for operating an electrosurgical instrument.

Electrosurgical systems and instruments therefor are known, for example, from U.S. Pat. No. 5,931,836 and DE 10 2009 041 329 A1. In the known systems, an electrosurgical instrument is connected to a generator in order to operate. DE 100 57 585 A1 describes an instrument in which instrument-specific configuration data are stored.

The existing systems, methods, instruments, and generators for operating electrosurgical instruments provide different advantages; however, additional improvements are desirable.

It is therefore the object of the present invention to provide an electrosurgical system, an electrosurgical instrument for use in an electrosurgical system, a method for operating an electrosurgical instrument, and a generator for operating an electrosurgical instrument, which are improved with respect to existing systems, methods, instruments, and generators.

This object is achieved via an electrosurgical system comprising an electrosurgical instrument, a generator which is configured for operating the electrosurgical instrument, a memory element in which at least one piece of operationally relevant information, which represents at least operating guide for the electrosurgical instrument, is stored, and a display which is configured to display the operationally relevant information, wherein the system is configured to transfer the operationally relevant information to the display.

The system according to the present invention is based inter alia on the finding that improvements may be achieved if operationally relevant information about an electrosurgical instrument is stored on a memory element of the system. In the systems known from the prior art, operating instructions are included with the instrument in paper form. Thus, the operating instructions in paper form are often perceived by the users of the systems as being a part of the delivery package, and are disposed of after unpacking the instrument. Alternatively, operating instructions are often archived. As a result of this approach, when they are needed, for example, during a clinical procedure, the operating instructions are not available at all, or only after searching the archive. The operationally relevant information must then either be rediscovered (in the case of disposal or loss) or requested from an archive. Both are time-consuming and pose a considerable risk to the patient under treatment. Missing or incorrectly organized operationally relevant information may result in incorrect operation of the instruments, which is also associated with considerable risk to the patient.

The system according to the present invention solves these problems and challenges in that the operationally relevant information is stored electronically within the system in a memory element of the system, and can be retrieved there immediately and at any time. The retrieved information is displayed on a display of the system and may thus be interpreted and used by a user of the system (for example, a treating physician). A mismatch may be safely avoided.

According to the present invention, the operationally relevant information is provided in electronic form in a memory element. This has the advantage that the manageability of the information is improved. Thus, for example, search functions and interactions may be made possible via a computer-based provision and preparation of the operationally relevant information for the display.

The system according to the present invention is preferably suitable for operating an electrosurgical handheld instrument.

The generator preferably comprises an ultrasonic generator including a generating unit for providing an excitation signal, via which the ultrasonic converter is able to generate an ultrasonic vibration. In addition or alternatively, the generator preferably comprises a high-frequency generator for providing a high-frequency signal to two output contacts of the generator. For operating the electrosurgical instrument, the generator provides an operating signal at its output contacts, which is processed by the electrosurgical instrument. The transmission of the operating signal to the instrument is carried out, for example, via a cable connection of the instrument. The cable connection preferably comprises a plug which may be plugged into the output contacts.

The operating signal preferably comprises the exciter signal and the high-frequency signal. By means of an ultrasonic converter present in the instrument, the exciter signal is converted into an ultrasonic vibration which may be used for treating the tissue of a patient.

According to the present invention, the high-frequency signal may be provided for monopolar and/or bipolar applications. In monopolar applications, the electrosurgical instrument has an electrode which is connected to one of the output contacts at which the high-frequency signal is provided. Simultaneously, the system comprises a patient electrode which is connected to another of the two output contacts at which the high-frequency signal is provided. While operating the electrosurgical instrument, the electrode is brought into contact with the tissue to be treated. The patient electrode is, for example, connected to a section of the patient's skin. In this way, the high-frequency signal generates a current which flows from the electrode, through a portion of the body, and back to the patient electrode.

In bipolar applications, the electrosurgical instrument has at least two electrodes, wherein each electrode is connected to one of the two output contacts at which the high-frequency signal is provided. In such a bipolar application, the electric current flows through the tissue which is between the two electrodes of the electrosurgical instrument and is in contact with them. The electrosurgical instrument may, for example, have two legs having one electrode each, or may have a rod-shaped applicator having two electrodes spaced apart in an axial direction.

If the high-frequency signal is provided in the microwave frequency range, the electrosurgical instrument may comprise a microwave antenna instead of one or more electrodes, or in addition thereto.

Within the scope of the present invention, the term “operationally relevant information” includes inter alia all tutorial and instructional information which relates to the intended and secure operation, use, usage, application, etc. of the electrosurgical instrument by a user. Operationally relevant information thus comprises, for example, all information which is found in printed operating instructions or instruction manuals, but which may preferably include additional information which cannot be provided in printed form, for example, animations or films.

Within the scope of the present invention, the term “memory element” is preferably to be understood to mean a permanent or semi-permanent electronic memory. Thus, the memory element may preferably comprise or be made up of a flash EEPROM (erasable programmable read-only memory). The operationally relevant information is stored in the memory element, preferably in the form of data. The display is preferably configured to interpret the data and to prepare it for the display.

The system according to the present invention relates to the application of electrosurgical instruments and generators, and methods for operating such instruments and generators. For understanding the present invention, the system according to the present invention will be described below with respect to a system made up of a combined ultrasonic and high-frequency generator, and an electrosurgical instrument driven by the generator.

However, the present invention is applicable in equal measure to electrosurgical systems, electrosurgical instruments, generators, and methods which use only ultrasound or only high frequency.

The system is preferably configured to transmit the operationally relevant information, while the display is linked to the memory element via signaling technology. The linking of the display to the memory element via signaling technology may be carried out in a wire-bound or cable-bound manner, or in a wireless or cable-free manner, preferably by means of near-field communication. The cable-bound linking may, for example, be carried out by means of the cable connection of the memory element, as soon as said cable connection is plugged via its plug into output contacts of the display or of a device which has an element forming the display. For near-field communication, the memory element may preferably comprise or may be made up of an RFID element. The operationally relevant information stored in the memory element may be read out of the RFID element (RFID: radio-frequency identification) by means of a radio interface of the display or of a device which has an element forming the display. The RFID element may be formed by an RFID transponder. In this case, the radio interface of the display or of the device preferably emits an electromagnetic alternating field which is influenced by the RFID transponder. By means of the radio interface, reflected waves of the electromagnetic alternating field are received, and information from the influence of the alternating field, which is stored in the RFID transponder, is gathered. In this way, the identification information may be transmitted by linking to the display via signaling technology.

Linking via signaling technology may be understood to be direct and indirect linking via signal technology. In the case of indirect linking via signaling technology, one element or multiple elements is/are preferably connected between the display and the memory element. Thus, the display may, for example, have a control unit which is directly linked to the memory element of the system according to the present invention (in this context, the display is indirectly linked to the memory element via the control unit).

Further preferably, the system according to the present invention comprises an operating unit (for example, a computer unit) for preparing, processing, browsing, and/or interacting with the displayed operationally relevant information.

According to one preferred embodiment of the system according to the present invention, the electrosurgical instrument includes the memory element. According to this embodiment, the memory element of the system according to the present invention thus forms an integral part of the electrosurgical instrument, which may be operated in the system.

The memory element is preferably arranged in a handheld portion of the electrosurgical instrument. Alternatively and/or in addition, the memory element is furthermore preferably arranged in the plug of the electrosurgical instrument. The arrangement in the plug of the electrosurgical instrument has the advantage that a shorter range is required for the data transfer between the memory element and the generator. In addition, a higher data rate may be achieved.

A significant advantage of this embodiment is that the operationally relevant information may be retrieved by the instrument itself. As a result, the operationally relevant information is available, regardless of the system in which, or the generator via which, the instrument is operated.

Preferably, only instrument-specific operationally relevant information is stored in the memory element of the instrument. Within the scope of the present invention, instrument-specific operationally relevant information may be understood to mean operating information which is relevant to the operation of the instrument type on which the operationally relevant information is stored. Operationally relevant information about other instrument types, for example, is not included, and may be stored on memory elements of these other instrument types.

Preferably, the operationally relevant information is transferred from the memory element of the electrosurgical instrument to the display, while the display is linked to the electrosurgical instrument via signaling technology. The linking of the display to the electrosurgical instrument via signaling technology may be carried out in a wire-bound or cable-bound manner, or in a wireless or cable-free manner, as described above.

According to another preferred embodiment of the electrosurgical system according to the present invention, the generator includes the memory element. According to this embodiment, the memory element of the system according to the present invention thus forms an integral part of the generator which may be used for operating the electrosurgical instrument.

Preferably, the operationally relevant information is transferred from the memory element of the generator to the display, while the display is linked to the generator via signaling technology. The linking of the generator to the electrosurgical instrument via signaling technology may be carried out in a wire-bound or cable-bound manner, or in a wireless or cable-free manner, as described above.

Further preferably, the storage of the operationally relevant information may be carried out before linking the generator to the display via signaling technology, by transferring the operationally relevant information to the generator, for example, within the scope of a field upgrade, in which the operationally relevant information is downloaded from a computer unit to the memory element of the generator via a data interface of the generator. After downloading, the data remain stored in the memory element of the generator as sleeper information, for example, in an encrypted form.

In another preferred embodiment of electrosurgical system according to the present invention, the generator includes the display for displaying the operationally relevant information. According to the present invention, the display of the system according to the present invention forms an integral part of the generator which may be used for operating the electrosurgical instrument.

A significant advantage of this embodiment is that a display which is already present in the generator may be used as a display for the operationally relevant information.

Preferably, according to this embodiment, the generator also has the features described above with respect to the present embodiment.

Further preferably, the operationally relevant information is transferred from the memory element of the electrosurgical instrument to the display, while the generator is linked to the electrosurgical instrument via signaling technology. The linking of the generator with the electrosurgical instrument via signaling technology may be carried out in a wire-bound or cable-bound manner, or in a wireless or cable-free manner, as described above.

In another preferred embodiment of the electrosurgical system according to the present invention, a separate display unit includes the display, or a medical information system includes the display. According to this embodiment, the display of the system according to the present invention forms an integral part of a separate display unit or an integral part of a medical information system.

If the display is configured as an integral part of a separate display unit, the advantage is achieved that a suitable display may be selected for the intended preparation of operationally relevant information for the system. Any shortcomings of a display integrated into the generator, for example, due to its small size, may thereby be overcome.

A suitable display for preparation of operationally relevant information is frequently to be found as an element of a medical information system which is present in a treatment room. According to this embodiment, the medical information system may be advantageously used to display the operationally relevant information.

Another preferred embodiment of the electrosurgical system according to the present invention includes a default language setting, wherein the system is configured to display the operationally relevant information by means of the display, in a language as a function of the default language setting.

Preferably, only the operationally relevant information which corresponds to a language according to the default language setting is transferred to the display. In this way, the data volume and, thus the duration of the retrieval of the information, is reduced.

According to a preferred refinement of the embodiment, the default language setting corresponds to a default language setting of the generator. Via this embodiment, the property of known generators, which display information in a predetermined language corresponding to a default language setting, is used in an advantageous manner. In this case, the default language setting of the generator is used to display the operationally relevant information in a language according to the default language setting.

According to a second aspect of the present invention, the initially specified object is achieved via a method for operating an electrosurgical instrument, comprising:
providing at least one piece of operationally relevant information, which represents at least one set of operating instructions for the electrosurgical instrument, to a memory element,
transferring the operationally relevant information to a display which is configured to display the operationally relevant information, and
displaying the operationally relevant information by means of the display.

Preferably, the operationally relevant information is transferred to the display while the memory element is linked via signaling technology to the display or to a device which includes the display.

According to one preferred embodiment of the method, the provision comprises storing the at least one piece of operationally relevant information in a memory element of the electrosurgical instrument.

One refinement of the preferred embodiment of the method comprises linking the electrosurgical instrument via signaling technology to a generator for operating the electrosurgical instrument, wherein the transfer comprises transferring the operationally relevant information to the generator.

In an additional preferred embodiment of the method, the provision comprises storing the at least one piece of operationally relevant information in a memory element of a generator for operating the electrosurgical instrument.

One refinement of the additional preferred embodiment of the method comprises linking the generator via signaling technology to the electrosurgical instrument, which includes a memory element in which identification information for identification of the electrosurgical instrument is stored, wherein the identification information is transferred to the generator;

identifying the electrosurgical instrument by means of the generator; and transferring the operationally relevant information for the identified electrosurgical instrument to the display.

According to this refinement, the operationally relevant information is stored in a memory element of the generator. Preferably, operationally relevant information for different instrument types is stored in the memory element of the generator. If the generator identifies the instrument, only operationally relevant information which is relevant to the identified instrument is transferred to the display.

According to another preferred embodiment of the method, the transfer of the operationally relevant information is carried out during the operation of the electrosurgical instrument.

The method according to the present invention and its refinements include steps which make them in particular suitable for being used by a system according to the present invention and its refinements. For the advantages, design variants, and embodiment details of the method and its refinements, reference will be made to the preceding description of the corresponding system features.

According to a third aspect of the present invention, the initially specified object is achieved via an electrosurgical instrument for use in an electrosurgical system, in particular an electrosurgical system of the type described above, wherein the electrosurgical instrument is configured to be operated by means of a generator, comprising a memory element in which at least one piece of operationally relevant information, which represents at least one set of operating instructions, is stored, wherein the instrument having a display is configured to be linkable via signaling technology for transferring the operationally relevant information to the display.

The electrosurgical instrument according to the present invention and its refinements have features which make them in particular suitable to be used for a system according to the present invention and in a method according to the present invention for operating an electrosurgical instrument and its refinements. With respect to advantages, design variants, and embodiment details of the electrosurgical instrument and its refinements, reference will be made to the preceding description of the corresponding system features and method features.

According to a fourth aspect of the present invention, the initially specified object is achieved via a generator for operating an electrosurgical instrument, comprising a memory element on which at least one piece of operationally relevant information, which represents at least one set of operating instructions for the electrosurgical instrument, is stored, wherein the generator having a display is configured to be linkable via signaling technology to the display for transmitting the operationally relevant information, or has a display for displaying the operationally relevant information.

One preferred embodiment of the generator includes the display for displaying the operationally relevant information.

The electrosurgical generator according to the present invention and its refinements have features which make them in particular suitable to be used for operating an electrosurgical instrument according to the present invention, for a system according to the present invention, in a method according to the present invention for operating an electrosurgical instrument and its refinements. With respect to advantages, design variants, and embodiment details of the generator and its refinements, reference will be made to the preceding description of the corresponding system features and method features.

Preferred embodiments of the present invention will be explained by way of example, based on the attached figures. The following are shown:

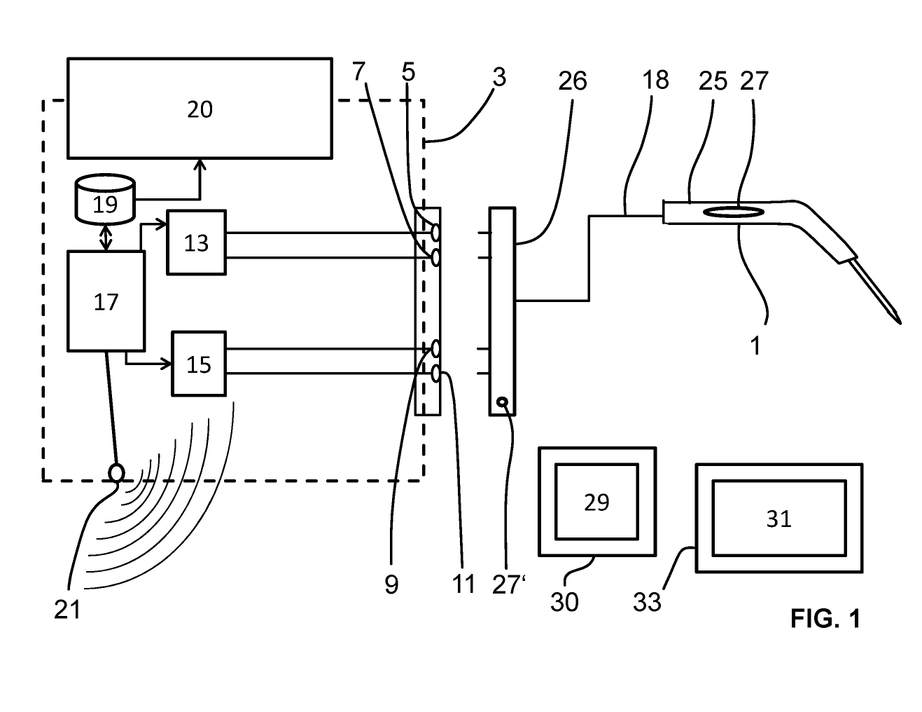

FIG. 1: a schematic representation of an exemplary embodiment of an electrosurgical system according to the present invention;

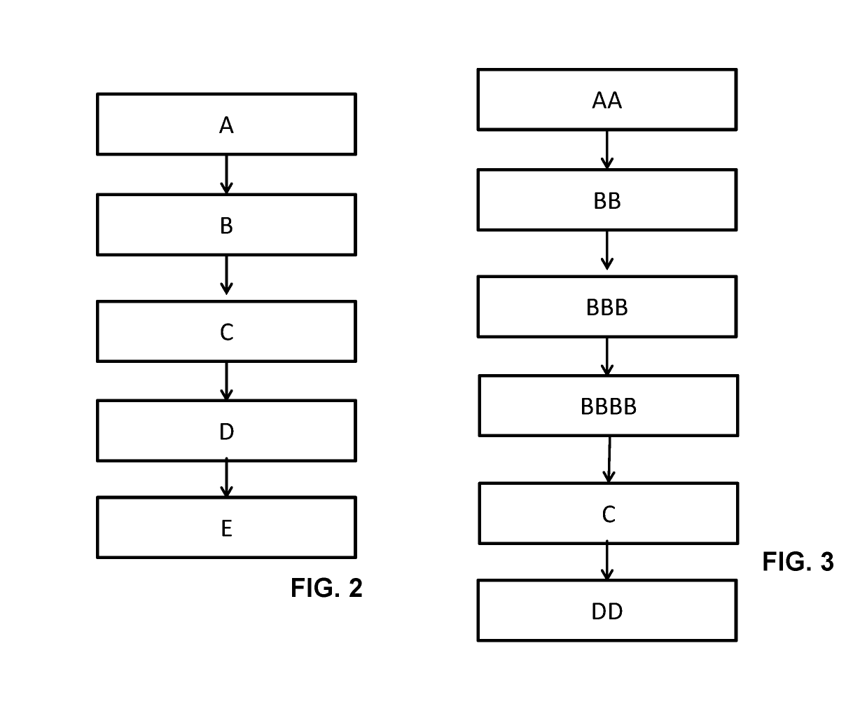

FIG. 2: a schematic flow chart of a first exemplary embodiment of a method according to the present invention; and

FIG. 3: a schematic flow chart of a second exemplary embodiment of a method according to the present invention.

FIG. 1 shows a generator 3 and an electrosurgical instrument 1 as an integral part of an electrosurgical system according to one exemplary embodiment of the present invention. FIG. 2 shows a schematic flow chart which depicts a method according to the present invention for operating an electrosurgical instrument according to a first exemplary embodiment. The method can be carried out by the system described with reference to FIG. 1.

An electrosurgical instrument 1 comprises a memory element 27 in which operationally relevant information is stored in a method step A. The operationally relevant information represents operating instructions for the electrosurgical instrument 1. In other words, the operationally relevant information is used for informing a user of the electrosurgical instrument 1, and indicates how the electrosurgical instrument 1 is to be used as intended.

In a method step B, the electrosurgical instrument 1 is linked to the generator 3 via signaling technology. For the linking via signaling technology, the generator 3 has a radio interface 21 by means of which the memory element 27, which is preferably arranged in a handheld portion 25 of the electrosurgical instrument 1, is read out. The memory element 27 preferably comprises an RFID transponder which is read out by means of the radio interface 21.

When reading out, in a method step D, the operationally relevant information is transferred to a control unit 17, from there further to a memory element 19, and from there further to a display 20 of the generator 3. Alternatively or in addition, in method step D, the operationally relevant information is transferred to a display 29 of a separate display unit 30 or a display 31 of a medical information system 33. In other words, in method step D, the operationally relevant information is transferred to the display 20, 29, and/or 31.

In a method step E, the operationally relevant information is displayed via the display 20, 29 and/or 31.

The generator 3 is configured to operate the electrosurgical instrument 1 in a method step C. The operation of the electrosurgical instrument 1 according to method step C may be carried out before, during, or after the transfer according to method step D. For operating the electrosurgical instrument 1, the generator provides an operating signal to multiple output contacts 5, 7, 9, and 11. The operating signal comprises an exciter signal at the output contacts 5 and 7, via which an ultrasonic converter may generate an ultrasonic vibration, and a high-frequency signal at the output contacts 9 and 11. The exciter signal is generated by an ultrasonic generator 13. The high-frequency signal is generated by a high-frequency generator 15.

For operation, the electrosurgical instrument 1 is connected via a cable connection 18 to the generator 3. On one end of the cable connection 18, a plug 26 is arranged which may preferably be plugged into the output contacts 5, 7, 9, and 11 via the first interface 12. The electrosurgical instrument 1 comprises an ultrasonic converter (not depicted), which generates an ultrasonic vibration for application in a tissue of the patient, from the exciter signal which is transferred via the cable connection 18 to the electrosurgical instrument 1.

In addition, the high-frequency signal is transferred via the cable connection 18 and is introduced into the tissue of the patient by means of the electrosurgical instrument 1. The ultrasonic generator 13 and the high-frequency generator 15 are controlled by a control unit 17.

The memory element 27 which is arranged in the handheld portion 25 of the electrosurgical instrument 1 may alternatively and/or additionally be arranged as a memory element 27′ in the plug 26 of the electrosurgical instrument 1.

FIG. 3 shows a schematic flow chart which depicts a method according to the present invention for operating an electrosurgical instrument according to a second exemplary embodiment. The method according to FIG. 3 may be carried out similarly to the method shown in FIG. 2. Identical method steps or method steps which are essentially functionally identical are provided with the same reference characters.

In a method step AA, operationally relevant information is downloaded to the generator 3 and stored by means of the memory element 19. The download is carried out, for example, by means of a computer which is not depicted, which is connected to the generator 3 via a data interface, for example, a USB interface (USB: universal serial bus). The operationally relevant information is initially stored (encrypted) only as sleeper information in the memory element 19.

In a method step BB, the electrosurgical instrument 1 is linked to the generator 3 via signaling technology. By means of the radio interface 21 of the generator 3, the memory element 27, 27′, on which identification information for identifying the electrosurgical instrument is stored, is read out. The identification information represents the instrument type. When reading out, the identification information is transferred to the control unit 17 in a method step BBB. In a method step BBBB, the control unit 17 identifies the electrosurgical instrument 1, in particular the instrument type, based on the identification information. In a method step DD, operationally relevant information about the identified instrument is transferred from the memory element 19 to the display 20, 29, and/or 31.

Alternatively to the manner described above, the transfer of the operationally relevant information (FIG. 2, method step D) and/or the identification information (FIG. 3, method step BBB) may also be carried out in a wire-bound manner. In this case, operationally relevant information and/or the identification information are, for example, transferred via the cable connection 18 between the instrument 1 and the generator 3. The cable connection 18 is formed from a cable which has a plug 26 and which is used during operation for transmitting the operating signal including the exciter signal and the high-frequency signal. The operationally relevant information and/or the identification information is/are, for example, modulated as an additional signal onto the high-frequency signal.

### REFERENCE CHARACTERS

1 Electrosurgical instrument
3 Generator
5, 7, 9, 11 Output contacts
13 Ultrasonic generator
15 High-frequency generator
17 Control unit
18 Cable connection
19 Memory element
20 Display
21 Radio interface
25 Handheld portion
26 Plug
27, 27′ Memory element
29 Display
30 Display unit
31 Display
33 Medical information system
A, B, C, D, E Method step
AA, BB, BBB, BBBB, DD Method step

1. An electrosurgical system, comprising
an electrosurgical instrument,
a generator which is configured for operating the electrosurgical instrument,
a memory element in which at least one piece of operationally relevant information, which represents at least one set of operating instructions for the electrosurgical instrument, is stored, and
a display which is configured to display the operationally relevant information,
wherein the system is configured to transfer the operationally relevant information to the display.

2. The electrosurgical system as claimed in claim 1,
wherein the electrosurgical instrument includes the memory element.

3. The electrosurgical system as claimed in claim 1,
wherein the generator includes the memory element.

4. The electrosurgical system as claimed in claim 1,
wherein the generator includes the display for displaying the operationally relevant information.

5. The electrosurgical system as claimed in claim 1,
wherein a separate display unit includes the display, or a medical information system includes the display.

6. The electrosurgical system as claimed in claim 1,
which has a default language setting,
wherein the system is configured to display the operationally relevant information by means of the display in a language as a function of the default language setting.

7. The electrosurgical system as claimed in claim 6,
wherein the default language setting corresponds to a default language setting of the generator.

8. A method for operating an electrosurgical instrument, comprising:
providing (A) at least one piece of operationally relevant information, which represents at least one set of operating instructions for the electrosurgical instrument, in a memory element,
transferring (D) the operationally relevant information to a display which is configured to display the operationally relevant information, and
displaying (E) the operationally relevant information by means of the display.

9. The method as claimed in claim 8, in which the provision (A) comprises storing at least one piece of operationally relevant information in a memory element of the electrosurgical instrument.

10. The method as claimed in claim 9, comprising linking (B) the electrosurgical instrument via signaling technology to a generator for operating the electrosurgical instrument, wherein the transfer comprises transferring the operationally relevant information to the generator.

11. The method as claimed in claim 8,
in which the provision comprises storing the at least one piece of operationally relevant information in the memory element of a generator for operating the electrosurgical instrument.

12. The method as claimed in claim 11, comprising:
linking the generator via signaling technology to the electrosurgical instrument, which includes a memory element in which identification information for identifying the electrosurgical instrument is stored, wherein the identification in formation information is transferred (BBB) to the generator,
identifying (BBBB) the electrosurgical instrument by means of the generator, and
transferring (DD) the operationally relevant information for the identified electrosurgical instrument to the display.

13. The method as claimed in claim 8,
transferring the operationally relevant information during the operation of the electrosurgical instrument.

14. An electrosurgical instrument for use in an electrosurgical system, in particular in an electrosurgical system as claimed in claim 1, wherein the electrosurgical instrument is configured to be operated by means of a generator, comprising
a memory element in which at least one piece of operationally relevant information, which represents at least one set of operating instructions for the electrosurgical instrument, is stored,
wherein the electrosurgical instrument is configured to be linkable via signaling technology to a display for transferring the operationally relevant information to the display.

15. A generator for operating an electrosurgical instrument, comprising
a memory element in which at least one piece of operationally relevant information, which represents at least one set of operating instructions for the electro-surgical instrument, is stored,
wherein the generator is configured to be linkable via signaling technology to a display for transferring the operationally relevant information to the display, or includes a display for displaying the operationally relevant information.
